(19) United States
(12) Patent Application Publication
(10) Pub. No.: US 2023/0225717 A1
(43) Pub. Date: Jul. 20, 2023

(54) GUIDEWIRE DELIVERY CATHETER

(71) Applicants: David NEUSTADTER, Nof Ayalon (IL); Boaz MANASH, Zichron Yaakov (IL); CARDIAC SUCCESS LTD., Yokneam (IL)

(72) Inventors: David NEUSTADTER, Nof Ayalon (IL); Boaz MANASH, Zichron Yaakov (IL)

(21) Appl. No.: 17/999,904

(22) PCT Filed: May 24, 2021
(86) PCT No.: PCT/IB2021/054492

§ 371 Date: Nov. 25, 2022

Related U.S. Application Data
(60) Provisional application No. 63/030,313, filed on May 27, 2020.

(51) Int. Cl. A61B 17/00

(52) U.S. Cl. CPC A61B 17/00234; A61B 2017/00331; A61B 2017/00358; A61M 2025/09183

(57) ABSTRACT

Embodiments of the present disclosure include a device for passing a guidewire around an anatomical structure within a body, comprising a catheter including an elongated tube having at least a first lumen and a second lumen, a guidewire configured to pass through the first lumen of the catheter, and a snare configured to pass through the second lumen of the catheter. A first exit of one of the first lumen and the second lumen may be positioned at or near a distal tip of the catheter, and a second exit of another of the first lumen and the second lumen may be positioned on a side of the catheter at a distance proximal to the distal tip of the catheter. A portion of the catheter distal to the second exit may be configured to bend around at least a portion of the anatomical structure of interest within the body.

### CROSS-REFERENCE TO RELATED APPLICATIONS

This application claims priority from U.S. Provisional Patent Application No. 63/030,313, filed May 27, 2020, which is fully incorporated herein by reference.

### TECHNICAL FIELD

The embodiments of the present disclosure relate in general to devices and methods involving a catheter for delivering a guidewire around one or more anatomical structures in a body. More specifically, the embodiments of the present disclosure relate to devices and methods of delivering and navigating a guidewire to at least partially surround anatomical structures in a body using a single catheter.

### BACKGROUND

Conventional transcatheter procedures exist for placing a guidewire within a body. There are numerous reasons for placing a guidewire in the body, including visualization of anatomical structures in fluoroscopy, or guidance of a catheter, tool, or implant along a particular path or to a particular location within the body. Guidewire placement within the arteries and into the chambers of the heart has been conventionally used in vascular catheterization procedures for many years for these purposes. Within the recent development of transcatheter structural heart procedures, there have been applications that require the placement of guidewires in more complex configurations. For example, there have been applications that require a guidewire to be placed around the leaflets of the mitral or tricuspid valves, around one or more papillary muscles within the ventricle of the heart, or through the spaces between the trabeculae of the ventricle.

However, placing a guidewire along a path around an anatomical structure within the heart often requires grasping the end of the guidewire with a snare, which generally requires the insertion of multiple catheters simultaneously, thereby significantly complicating the procedure. Therefore, there is a need for a device and method of delivering and navigating a guidewire around anatomical structures using a single catheter.

### SUMMARY

Presently disclosed embodiments recognize that a need exists for improved devices and methods for delivering and navigating a guidewire around anatomical structures within the body. The embodiments of the present disclosure include devices and methods of delivering and navigating a guidewire around anatomical structures within the body, for example to a ventricle of a heart. Advantageously, the exemplary embodiments provide devices and methods of delivering and navigating a guidewire around anatomical structures within the body, such as a ventricle of a heart, using a single catheter. Various embodiments of the disclosure may include one or more of the following aspects.

Consistent with an embodiment of the present disclosure, a device for passing a guidewire around an anatomical structure of interest within a body is provided. The device may comprise a catheter including an elongated tube having at least a first lumen and a second lumen, a guidewire configured to pass through the first lumen of the catheter, and a snare configured to pass through the second lumen of the catheter. A first exit of one of the first lumen and the second lumen may be positioned at or near a distal tip of the catheter, and a second exit of another of the first lumen and the second lumen may be positioned on a side of the catheter at a distance proximal to the distal tip of the catheter. A portion of the catheter distal to the second exit may be configured to bend around at least a portion of the anatomical structure of interest within the body.

In some embodiments, the first lumen may be a central lumen of the catheter with the first exit positioned at or near the distal tip of the catheter, and the second lumen may be a side lumen of the catheter with the second exit positioned on the side of the catheter at the distance proximal to the distal tip of the catheter. In other embodiments, the second lumen may be a central lumen of the catheter with the first exit positioned at or near the distal tip of the catheter, and the first lumen may be a side lumen of the catheter with the second exit positioned on the side of the catheter at the distance proximal to the distal tip of the catheter.

In some embodiments, the second exit may be positioned between about 2 cm and about 20 cm proximal to the distal tip of the catheter. In some embodiments, the second exit may be positioned between about 5 cm and about 15 cm proximal to the distal tip of the catheter. In other embodiments, the portion of the catheter distal to the second exit may include at least one of a pre-shaped portion, a flexible portion, a steerable portion, or a portion with bending properties. In yet another embodiment, the portion of the catheter distal to the second exit may include at least one of a metal wire braid, a metal wire coil, a cut hypotube, a non-metal braid, a pullwire, a pre-shaped polymer, or a pre-shaped shape-memory component.

In some embodiments, a portion of the catheter between the first exit and the second exit may be configured to position the first exit and the second exit such that the guidewire and the snare exiting the first and second lumens engage each other. In other embodiments, the snare may be pre-loaded within the second lumen of the catheter, and a handle of the catheter may include a mechanism for exposing, positioning, and retracting the snare. Additionally, or alternatively, the snare may include a snare sleeve and a snare wire within the snare sleeve, and the mechanism for exposing, positioning, and retracting the snare may include a first slider configured to control a movement of the snare sleeve and a second slider configured to control a movement of the snare wire. In some embodiments, the mechanism for exposing, positioning, and retracting the snare may include a sliding U-shaped tube.

In some embodiments, the guidewire may be pre-loaded within the first lumen of the catheter, and a handle of the catheter may include a mechanism for storing a length of the guidewire within the handle. In some embodiments, a distal portion of the guidewire may be pre-shaped to facilitate engaging a distal end of the guidewire with the snare. In other embodiments, the snare may include at least one of a snare sleeve or a snare wire, and a distal portion of at least one of the snare wire or the snare sleeve may be pre-shaped to facilitate engaging a distal end of the guidewire with the snare. In yet another embodiment, the device may further comprise an inner catheter configured to pass through a central lumen of the catheter, the guidewire or the snare may be configured to pass through a lumen of the inner catheter, a distal end of the inner catheter may extend past the distal tip of the catheter, and the distal end of the inner catheter may be configured to aid in positioning a distal end of the guidewire or the snare. In some embodiments, the distal end of the inner catheter may be pre-shaped. Additionally, or alternatively, the distal end of the inner catheter may be steerable.

In some embodiments, the mechanism for exposing, positioning, and retracting the snare may be configured such that the snare wire travels through a channel with an inner diameter of less than 10 mm. In other embodiments, the mechanism for exposing, positioning, and retracting the snare may be configured such that the snare wire travels through a channel with an inner diameter of less than 6 mm. Additionally, or alternatively, the mechanism for exposing, positioning, and retracting the snare may be configured such that the snare wire travels through a channel comprising an outer tube and an inner tube, and the outer tube may be configured to slide over the inner tube.

Additional objects and advantages of the embodiments will be set forth in part in the description that follows, and in part will be obvious from the description or may be learned by practice of the embodiments. The objects and advantages of the embodiments will be realized and attained by means of the elements and combinations particularly pointed out in the appended claims.

It is to be understood that both the foregoing general description and the following detailed description are exemplary and explanatory only and are not restrictive of the claims.

### BRIEF DESCRIPTION OF THE DRAWINGS

Some features of disclosed embodiments are set forth with particularity in the claims that follow. Additional details of the features and advantages of the disclosed embodiments will be obtained by reference to the following detailed description that sets forth illustrative examples, in which the disclosed principles are utilized, and the accompanying drawings of which:

FIG. 1 illustrates an exemplary catheter for delivering a guidewire, consistent with an embodiment of the present disclosure;

FIG. 2 illustrates one embodiment of an exemplary device for delivering a guidewire, consistent with an embodiment of the present disclosure;

FIG. 3 illustrates another embodiment of the exemplary device for delivering a guidewire of FIG. 2, consistent with an embodiment of the present disclosure;

FIG. 4 illustrates an exemplary guidewire, consistent with an embodiment of the present disclosure;

FIG. 5A illustrates another embodiment of an exemplary device for delivering a guidewire, consistent with an embodiment of the present disclosure;

FIG. 5B illustrates an embodiment of a cross-sectional view of the exemplary device for delivering a guidewire of FIG. 5A, consistent with an embodiment of the present disclosure;

FIG. 5C illustrates another embodiment of a cross-sectional view of the exemplary device for delivering a guidewire of FIG. 5A, consistent with an embodiment of the present disclosure;

FIG. 6 illustrates another embodiment of the exemplary device for delivering a guidewire of FIG. 5A, consistent with an embodiment of the present disclosure;

FIG. 7A illustrates the exemplary device for delivering a guidewire of FIG. 5A within a ventricle of a heart at a first stage of a procedure, consistent with an embodiment of the present disclosure;

FIG. 7B illustrates the exemplary device for delivering a guidewire of FIG. 5A within a ventricle of a heart at a second stage of a procedure, consistent with an embodiment of the present disclosure;

FIG. 8A illustrates an exemplary snare mechanism, consistent with an embodiment of the present disclosure;

FIG. 8B illustrates a first step of manipulating the exemplary snare mechanism of FIG. 8A, consistent with an embodiment of the present disclosure;

FIG. 8C illustrates a second step of manipulating the exemplary snare mechanism of FIG. 8A, consistent with an embodiment of the present disclosure;

FIG. 8D illustrates an exemplary device for delivering a guidewire after performing the second step of FIG. 8C, consistent with an embodiment of the present disclosure;

FIG. 9A illustrates a third step of manipulating the exemplary snare mechanism of FIG. 8A, consistent with an embodiment of the present disclosure;

FIG. 9B illustrates an exemplary device for delivering a guidewire after performing the third step of FIG. 9A, consistent with an embodiment of the present disclosure;

FIG. 9C illustrates a fourth step of manipulating the exemplary snare mechanism of FIG. 8A, consistent with an embodiment of the present disclosure;

FIG. 9D illustrates an exemplary device for delivering a guidewire after performing the fourth step of FIG. 9C, consistent with an embodiment of the present disclosure;

FIG. 10A illustrates a fifth step of manipulating the exemplary snare mechanism of FIG. 8A, consistent with an embodiment of the present disclosure;

FIG. 10B illustrates an exemplary device for delivering a guidewire after performing the fifth step of FIG. 10A, consistent with an embodiment of the present disclosure;

FIG. 10C illustrates a sixth step of manipulating the exemplary snare mechanism of FIG. 8A, consistent with an embodiment of the present disclosure;

FIG. 10D illustrates an exemplary device for delivering a guidewire after performing the fifth step of FIG. 10C, consistent with an embodiment of the present disclosure;

FIG. 11A illustrates another exemplary snare mechanism, consistent with an embodiment of the present disclosure;

FIG. 11B illustrates a first step of manipulating the exemplary snare mechanism of FIG. 11A, consistent with an embodiment of the present disclosure;

FIG. 11C illustrates an exemplary device for delivering a guidewire after performing the first step of FIG. 11B, consistent with an embodiment of the present disclosure;

FIG. 12A illustrates a second step of manipulating the exemplary snare mechanism of FIG. 11A, consistent with an embodiment of the present disclosure;

FIG. 12B illustrates an exemplary device for delivering a guidewire after performing the second step of FIG. 12A, consistent with an embodiment of the present disclosure;

FIG. 13A illustrates a third step of manipulating the exemplary snare mechanism of FIG. 11A, consistent with an embodiment of the present disclosure;

FIG. 13B illustrates an exemplary device for delivering a guidewire after performing the third step of FIG. 13A, consistent with an embodiment of the present disclosure;

FIG. 14A illustrates a fourth step of manipulating the exemplary snare mechanism of FIG. 11A, consistent with an embodiment of the present disclosure;

FIG. 14B illustrates an exemplary device for delivering a guidewire after performing the fourth step of FIG. 14A, consistent with an embodiment of the present disclosure;

FIG. 14C illustrates a fifth step of manipulating the exemplary snare mechanism of FIG. 11A, consistent with an embodiment of the present disclosure;

FIG. 14D illustrates an exemplary device for delivering a guidewire after performing the fifth step of FIG. 14C, consistent with an embodiment of the present disclosure;

FIG. 15 illustrates various embodiments of an exemplary snare, consistent with an embodiment of the present disclosure;

FIG. 16 illustrates an exemplary snare mechanism, consistent with an embodiment of the present disclosure; and

FIG. 17 illustrates another exemplary snare mechanism, consistent with an embodiment of the present disclosure.

### DETAILED DESCRIPTION OF EMBODIMENTS

The present disclosure relates to methods and devices for delivering and navigating a guidewire around an anatomical structure within a body. While the present disclosure provides examples of delivering and navigating a guidewire to a ventricle of a heart and around a plurality of papillary muscles using a single catheter, it should be noted that aspects of the disclosure in their broadest sense, are not limited to devices for manipulation around papillary muscles. Rather, it is contemplated that the forgoing principles may be applied to other devices for delivering and navigating a guidewire around any anatomical structure within a body. In addition, the term “guidewire” refers generally to any element that is capable of extending, encircling, and/or looping around an object, such as a muscle in the body. For example, a guidewire may be a wire, a string, a pullwire, a tube, an inflatable tube, a sling, or any elongated tool capable of bending around an anatomical structure within a body. Moreover, as used herein, the term “snare” may generally refer to any mechanism that is capable of being mounted on a wire or a thin tube (for example, at a distal end thereof) and that can be used to capture and grasp a guidewire.

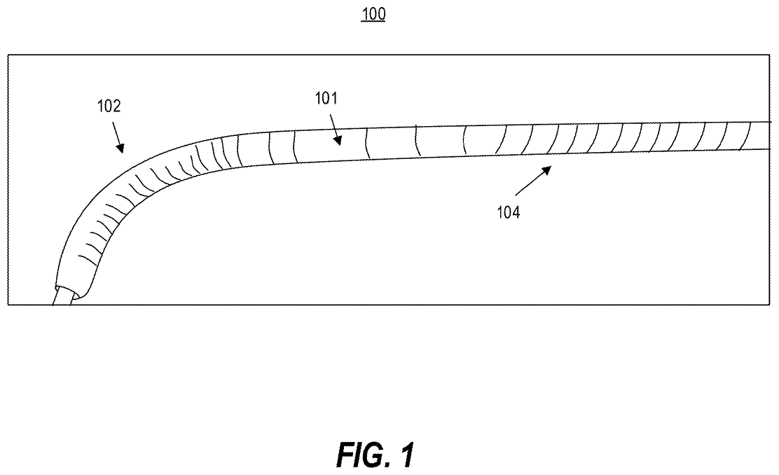

Referring to FIG. 1, an exemplary device 100 for delivering a guidewire to an anatomical structure, such as a heart, within a body, consistent with the present disclosure, may include a catheter 101 comprising an elongated tube with a first portion 102 and a second portion 104. In some embodiments, the first portion 102 may include a pre-shaped portion. Accordingly, as shown in FIG. 1, the first portion 102 may be pre-shaped to bend in a predefined direction. In other embodiments, the first portion 102 may include a flexible portion, a steerable portion, or a portion with bending properties such that the first portion 102 can navigate around an anatomical structure within a body. In some embodiments, the first portion 102 may include a metal wire braid, a metal wire coil, a cut hypotube, a non-metal braid, a pullwire, a pre-shaped polymer, or a pre-shaped shape-memory component. A pullwire may refer generally to any element that is capable of extending through a tube, such as a string, a strand, a wire, a coil, or a tube. The second portion 104 may include a highly flexible portion that is capable of navigating around or encircling at least a portion of an anatomical structure within a body. Similar to the first portion 102, the second portion 104 may include a flexible portion, a steerable portion, or a portion with bending properties such that the second portion 104 can navigate around an anatomical structure within a body. In some embodiments, the second portion 104 may include a metal wire braid, a metal wire coil, a cut hypotube, a non-metal braid, a pullwire, a pre-shaped polymer, or a pre-shaped shape-memory component. In some embodiments, the second portion 104 may be more flexible than the first portion 102.

In some embodiments, device 100 may be configured to allow placement of a guidewire around an anatomical structure within a cavity, such as a heart, using a single catheter 101. As discussed below, catheter 101 may comprise at least two lumens. In some embodiments, a guidewire may be configured to pass through a first lumen of catheter 101, and a snare may be configured to pass through a second lumen of catheter 101. The snare may be configured to grasp a distal end of the guidewire outside of catheter 101 and pull the distal end of the guidewire into the distal end of the second lumen, thereby forming a loop around an anatomical structure. In some embodiments, catheter 101 may comprise more than two lumens, such as three lumens, four lumens, or five lumens. In some embodiments, catheter 101 may comprise a plurality of guidewires configured to pass through a plurality of lumens of catheter 101 and may further comprise a plurality of snares configured to pass through a plurality of lumens of catheter 101.

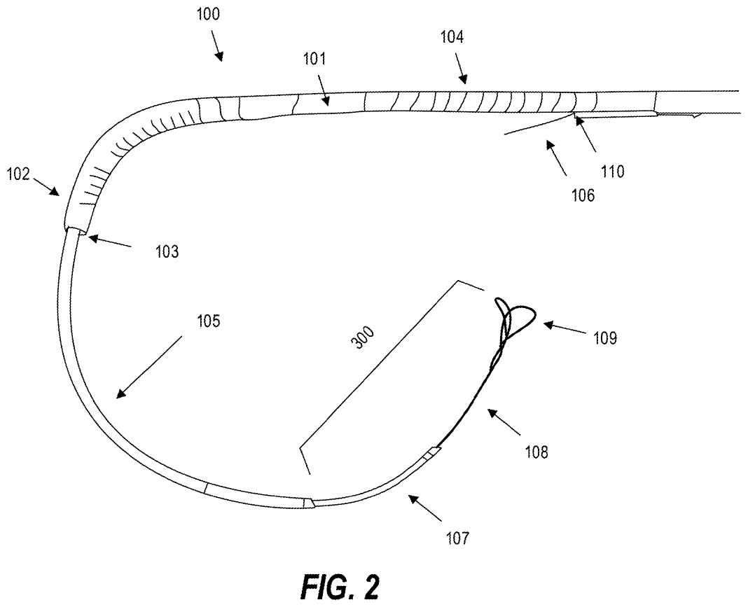

Referring now to FIG. 2, one embodiment of the exemplary device 100 for delivering a guidewire, consistent with an embodiment of the present disclosure, is shown. As discussed above, device 100 may include a catheter 101 comprising an elongated tube with a first portion 102 and a second portion 104. As discussed above, catheter 101 may comprise at least two lumens. In some embodiments, device 100 may further include a guidewire 106 configured to pass through a first lumen of catheter 101. Additionally, device 100 may include a snare 300 comprising a snare sleeve 107, a snare wire 108, and a snare tip 109. Snare 300 may be configured to pass through a second lumen of catheter 101.

As illustrated in FIG. 2, catheter 101 may further include a first exit 103 of one of the first lumen and the second lumen positioned at or near a distal tip of catheter 101. Additionally, catheter 101 may include a second exit 110 of the other of the first lumen and the second lumen positioned on a side of catheter 101. The second exit 110 may be positioned at a distance proximal to the distal tip of the catheter. By way of example, the second exit 110 may be positioned between about 2 cm and about 20 cm from the distal tip of catheter 101. In other embodiments, the second exit 110 may be positioned between about 5 cm and about 15 cm from the distal tip of catheter 101. As shown in FIG. 2, in some embodiments, the second lumen, through which snare 300 is configured to pass, may be a central lumen of catheter 101 with the first exit 103 positioned at or near the distal tip of catheter 101. Additionally, the first lumen, through which guidewire 106 is configured to pass, may be a side lumen of catheter 101 with the second exit 110 positioned on the side of catheter 101 at a distance proximal to the distal tip of catheter 101.

In some embodiments, a portion of catheter 101 distal to the second exit 110 may be configured to bend around at least a portion of an anatomical structure of interest within a body. For example, as discussed above, the first portion 102 and/or the second portion 104 of catheter 101 may include a flexible portion, a steerable portion, or a portion with bending properties such that the portion of catheter 101 distal to the second exit 110 can bend around at least a portion of the anatomical structure that guidewire 106 is intended to be placed around. In some embodiments, device 100 may further include an inner catheter 105 configured to pass through a lumen of catheter 101. For example, inner catheter 105 may be configured to pass through a first lumen or a second lumen of catheter 101.

In some embodiments, the first exit 103 may be positioned at or near the distal tip of catheter 101 such that, when the first portion 102 and/or the second portion 104 of catheter 101 is positioned around the anatomical structure of interest, the distal tip of catheter 101, or the distal tip of inner catheter 105, may guide snare 300 inserted therethrough to be positioned near the second exit 110. Such positioning of snare 300 near the second exit 110 may facilitate the use of snare 300 to grasp a distal end of guidewire 106 exiting the second exit 110 of catheter 101. Additionally, or alternatively, at least one of inner catheter 105, snare 300, or snare sleeve 107 may be pre-shaped in order to aid in positioning snare 300 near the second exit 110 of catheter 101. In some embodiments, for example, inner catheter 105 may be steerable to aid in positioning snare 300 near the second exit 110 of catheter 101.

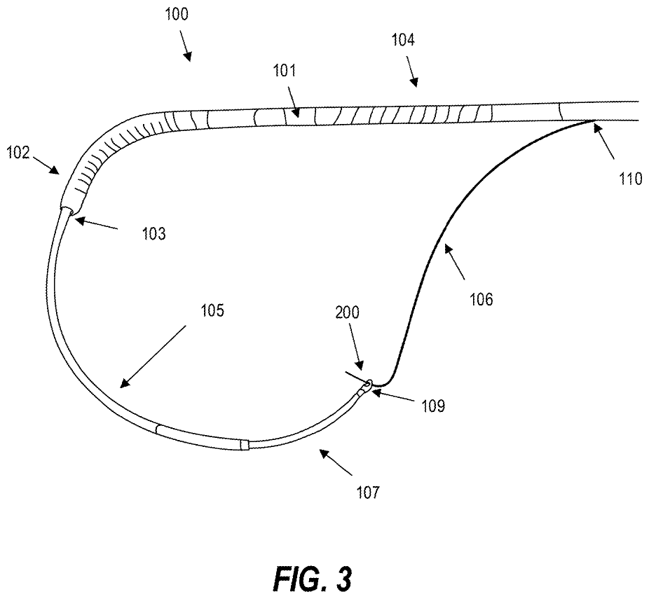

As discussed above, positioning snare 300 near the second exit 110 may facilitate the use of snare 300 to grasp a distal end of guidewire 106 exiting the second exit 110 of catheter 101. By way of example, as illustrated in FIG. 3, when snare 300 is brought near the second exit 110 of catheter 101, snare tip 109 of snare 300 may be configured to grasp a distal end 200 of guidewire 106 exiting the second exit 110 of catheter 101. Accordingly, snare tip 109 may grasp the distal end 200 of guidewire 106 and guide guidewire 106 around an anatomical structure within a body.

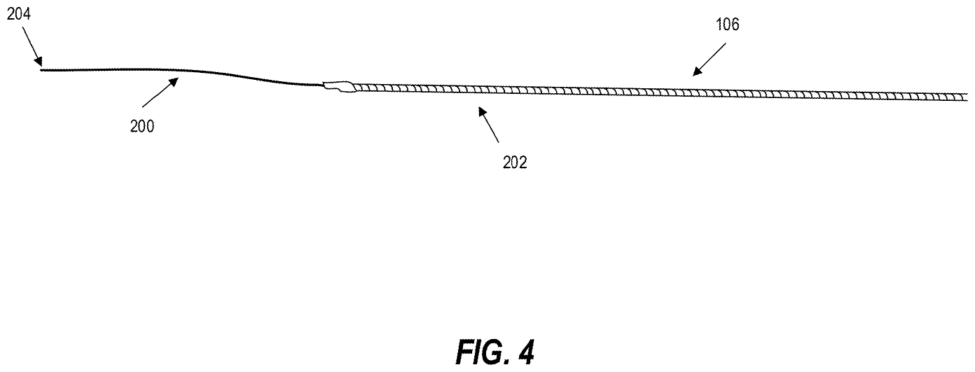

In some embodiments, guidewire 106 may be flexible so as to easily pass along the intended path around an anatomical structure of interest. Additionally, or alternatively, guidewire 106 may be sufficiently rigid such that guidewire 106 can be pushed through a lumen of catheter 101. FIG. 4 illustrates an embodiment of a guidewire 106, consistent with the embodiments of the present disclosure. As illustrated in FIG. 4, guidewire 106 may include a distal end 200 and proximal portion 202. In some embodiments, distal end 200 of guidewire 106 may be configured to be highly flexible such that distal end 200 can be easily grasped and firmly held, for example by snare tip 109 of FIG. 3. In some embodiments, distal end 200 of guidewire 106 may further include a rigid tip 204. Rigid tip 204 may be configured to prevent guidewire 106 from slipping out of snare tip 109 of snare 300 after distal end 200 has been grasped by snare tip 109. In some embodiments, distal end 200, rigid tip 204, and/or proximal portion 202 of guidewire 106 may be radiopaque so as to aid in visualization while snare tip 109 is grasping distal end 200. In some embodiments, distal end 200, rigid tip 204, and/or proximal portion 202 of guidewire 106 may be manufactured from stainless steel or nitinol. Additionally, or alternatively, distal end 200, rigid tip 204, and/or proximal portion 202 of guidewire 106 may be made of a metal cable, such as a twisted metal cable. In some embodiments, distal end 200, rigid tip 204, and/or proximal portion 202 of guidewire 106 may be coated with polytetrafluoroethylene (PTFE).

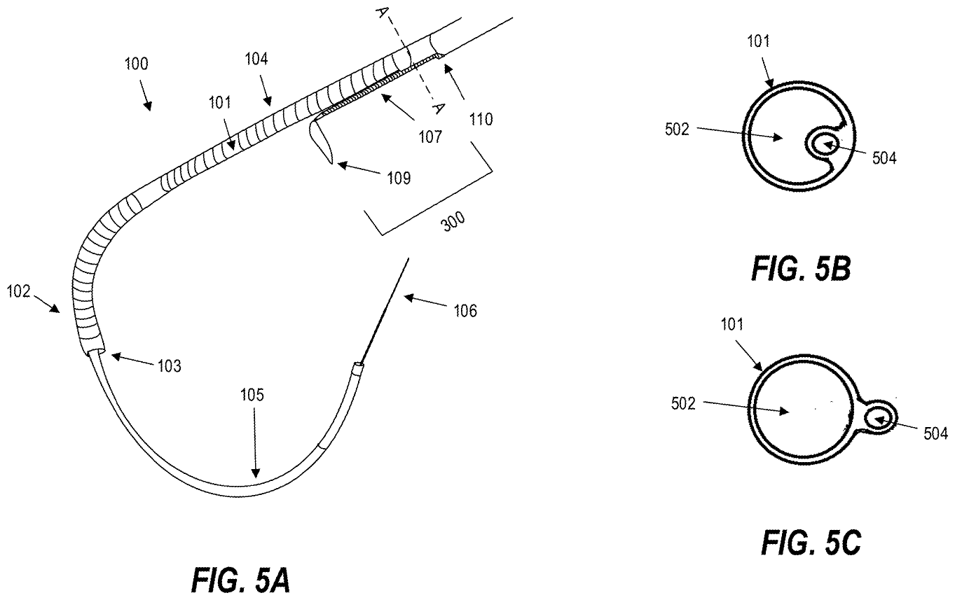

Referring now to FIGS. 5A-5C, another embodiment of device 100 for delivering a guidewire, consistent with an embodiment of the present disclosure, is shown. As discussed above, device 100 may include a catheter 101 comprising an elongated tube with a first portion 102 and a second portion 104. Catheter 101 may comprise at least two lumens. By way of example, FIGS. 5B and 5C illustrate two embodiments of a cross-sectional view of catheter 101 taken along the dashed line A-A in FIG. 5A. As illustrated in FIGS. 5B and 5C, catheter 101 may comprise at least a first lumen 502 and a second lumen 504. In some embodiments, device 100 may further include a guidewire 106 configured to pass through a first lumen of catheter 101, such as first lumen 502 of FIGS. 5B and 5C. Additionally, device 100 may include a snare 300 comprising a snare sleeve 107, a snare wire (not shown) such as snare wire 108 of FIG. 2, and a snare tip 109. Snare 300 may be configured to pass through a second lumen of catheter 101, such as second lumen 504 of FIGS. 5B and 5C.

As illustrated in FIG. 5A, catheter 101 may further include a first exit 103 of one of the first lumen and the second lumen positioned at or near a distal tip of catheter 101. Additionally, catheter 101 may include a second exit 110 of the other of the first lumen and the second lumen positioned on a side of catheter 101. The second exit 110 may be positioned at a distance proximal to the distal tip of the catheter. By way of example, the second exit 110 may be positioned between about 2 cm and about 20 cm from the distal tip of catheter 101. In other embodiments, the second exit 110 may be positioned between about 5 cm and about 15 cm from the distal tip of catheter 101. As shown in FIGS. 5A-5C, in some embodiments, second lumen 504, through which snare 300 may be configured to pass, may be a side lumen of catheter 101 with the second exit 110 positioned on the side of catheter 101 at a distance proximal to the distal tip of catheter 101. Additionally, first lumen 502, through which guidewire 106 may be configured to pass, may be a central lumen of catheter 101 with the first exit 103 positioned at or near the distal tip of catheter 101.

In some embodiments, a portion of catheter 101 distal to the second exit 110 may be configured to bend around at least a portion of an anatomical structure of interest within a body. For example, as discussed above, the first portion 102 and/or the second portion 104 of catheter 101 may include a flexible portion, a steerable portion, or a portion with bending properties such that the portion of catheter 101 distal to the second exit 110 can bend around at least a portion of the anatomical structure that guidewire 106 is intended to be placed around. In some embodiments, device 100 may further include an inner catheter 105 configured to pass through a lumen of catheter 101. For example, inner catheter 105 may be configured to pass through a first lumen or a second lumen of catheter 101.

In some embodiments, the second exit 110 may be positioned at a location on catheter 101 such that, when the first portion 102 and/or the second portion 104 of catheter 101 is positioned around the anatomical structure of interest, the distal tip of catheter 101, or the distal tip of inner catheter 105, may guide guidewire 106 inserted therethrough to be positioned near the second exit 110. Such positioning of guidewire 106 near the second exit 110 may facilitate the use of snare 300 protruding from the second exit 110 to grasp a distal end of guidewire 106 protruding from the first exit 103 of catheter 101. Additionally, or alternatively, at least one of inner catheter 105, guidewire 106, and/or snare 300 may be pre-shaped in order to aid in positioning guidewire 106 near the second exit 110 of catheter 101. In some embodiments, for example, inner catheter 105 may be steerable to aid in positioning guidewire 106 near the second exit 110 of catheter 101 and to aid in grasping the tip of guidewire 106 with snare tip 109 of snare 300.

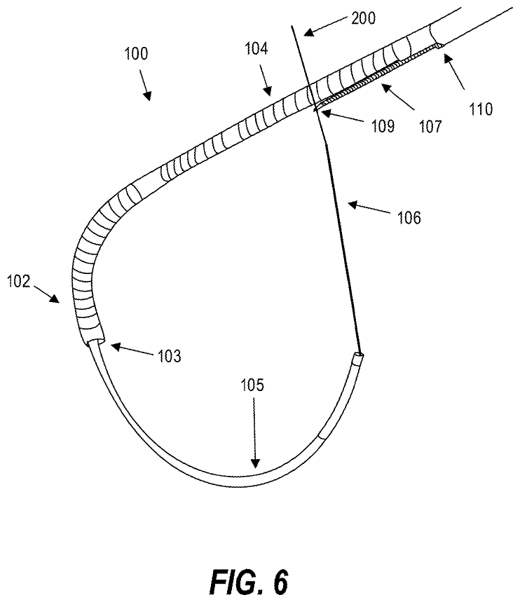

As discussed above, positioning guidewire 106 near the second exit 110 may aid in grasping the tip of guidewire 106 with snare 300. By way of example, as illustrated in FIG. 6, when guidewire 106 is brought near the second exit 110 of catheter 101, snare tip 109 of snare 300 protruding from the second exit 110 may be configured to grasp a distal end 200 of guidewire 106 protruding from the first exit 103 of catheter. Accordingly, snare tip 109 may grasp the distal end 200 of guidewire 106 and pull guidewire 106 into the second lumen through second exit 110.

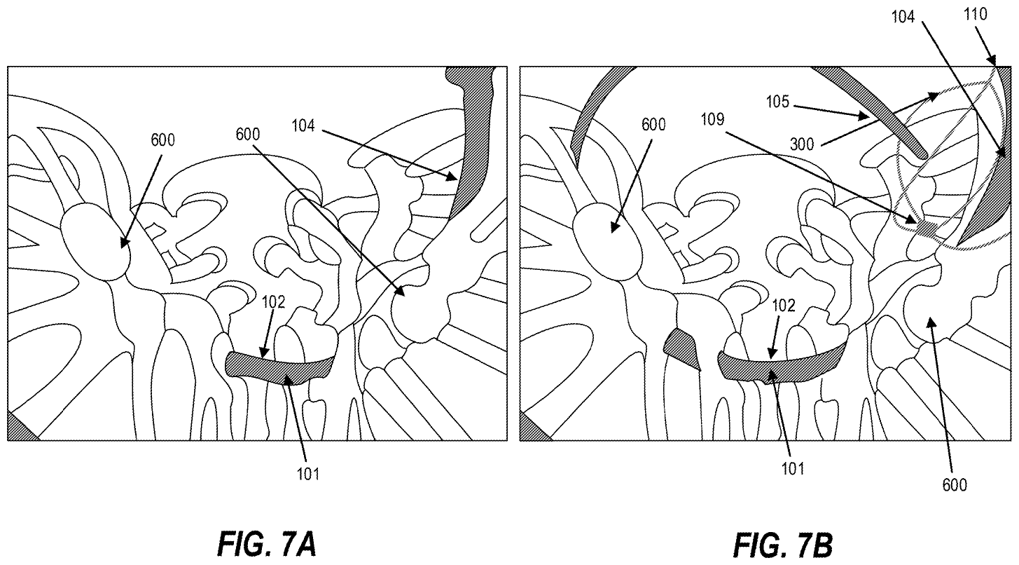

FIGS. 7A and 7B illustrate exemplary device 100 of FIG. 5A located within a ventricle of a heart at a first stage and at a second stage of a procedure, consistent with the embodiments of the present disclosure. By way of example, FIG. 7A illustrates first portion 102 and second portion 104 of catheter 101 positioned and bent around papillary muscles 600 in a ventricle of a heart. FIG. 7B illustrates first portion 102 and second portion 104 of catheter 101 positioned and bent around papillary muscles 600. FIG. 7B also illustrates a distal tip of inner catheter 105 positioned in the vicinity of snare 300, which is protruding from the second exit 110 of catheter 101 at a location proximal to the distal tip of catheter 101.

In some embodiments, snare 300 may be pre-loaded in the side lumen of catheter 101. Accordingly, in some embodiments, snare 300 may not need to be removed completely from the side lumen of catheter 101 throughout a procedure, such as the procedure illustrated in FIGS. 7A and 7B. Referring back to FIGS. 5 and 6, in some embodiments, snare 300 may be pre-loaded in the side lumen of catheter 101, extended out the second exit 110 of catheter 101 to grasp a distal tip 200 of guidewire 106, pulled back into the side lumen of catheter 101 via the second exit 110 to firmly hold guidewire 106 as catheter 101 is removed from the body. As catheter 101 is being removed from the body, snare 300 may grasp and hold onto guidewire 106, thereby pulling guidewire 106 out of the body with catheter 101. When outside the body, snare 300 may be configured to extend out through the second exit 110 again to release guidewire 106 from snare 300.

In some embodiments, snare 300 may be configured to remain within the side lumen of catheter 101. In other embodiments, snare 300 may be positioned within the side lumen of catheter 101 and may include a snare wire, such as snare wire 108 of FIG. 2, and snare tip 109 connected to a distal end of the snare wire. Snare 300 may further include snare sleeve 107, through which the snare wire may run. The snare sleeve 107 may be moved proximally and distally relative to the snare wire so as to cover or expose snare tip 109. In some embodiments, the proximal ends of the snare wire, such as snare wire 108 of FIG. 2, and snare sleeve 107 may be configured to exit a proximal end of the side lumen of catheter 101 into a handle (not shown) of catheter 101.

In some embodiments of the present disclosure, catheter 101 may include a handle, and the handle of catheter 101 may include a snare mechanism. The snare mechanism may be configured to move the snare wire, such as snare wire 108 of FIG. 2, and/or snare sleeve 107 in a proximal and/or distal direction so as to manipulate the snare wire 108 and/or snare sleeve 107 to grasp the distal tip 200 of guidewire 106. Additionally, or alternatively, the snare mechanism may be configured to rotate snare wire 108 in order to orient snare tip 109 and facilitate capturing or grasping the distal tip 200 of guidewire 106 with snare tip 109.

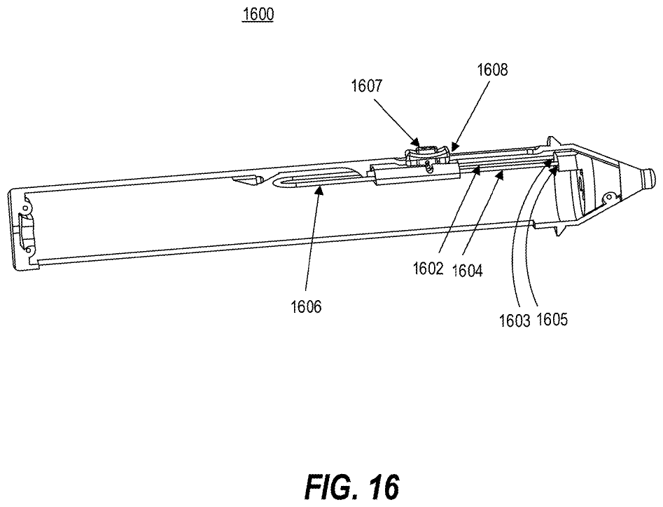

In some embodiments, as illustrated in FIG. 16, a snare mechanism 1600 may be provided to manipulate a snare, such as snare 300. In some embodiments, the snare mechanism 1600 may include two straight tubes 1602, 1604 and one U-shaped tube 1606. The straight legs of the U-shaped tube 1606 may be configured to slide either within or over the two straight tubes 1602, 1604 of the snare mechanism 1600. Snare wire 108 may run through one of the two straight tubes, such as straight tube 1602, and a free end 1603 of the first straight tube 1602 may be affixed to a handle of catheter 101 at a location where snare wire 108 enters the handle. Snare wire 108 may further run through the U-shaped tube 1606 and through the second straight tube of the two straight tubes, such as straight tube 1604, and may be affixed to a free end 1605 of the second straight tube 1604, which may also be affixed to the handle of catheter 101. The U-shaped tube 1606 may be configured to slide in a proximal and/or distal direction by a snare actuator 1608, thereby pushing and/or pulling snare wire 108 and causing snare 300 to move in a proximal and/or distal direction. Such configuration may cause snare 300 to move twice the distance that the U-shaped tube 1606 may move, thereby reducing the range of motion needed for the snare actuator 1608 in the handle by a factor of two relative to the range of motion of snare 300 itself. Additionally, or alternatively, the snare mechanism 1600 may be configured to lock snare 300 in a fully retracted position, thereby requiring the release of the lock in order to open snare 300. For example, a lock release button 1607 may be pressed to release snare 300 from a locked position and open snare 300.

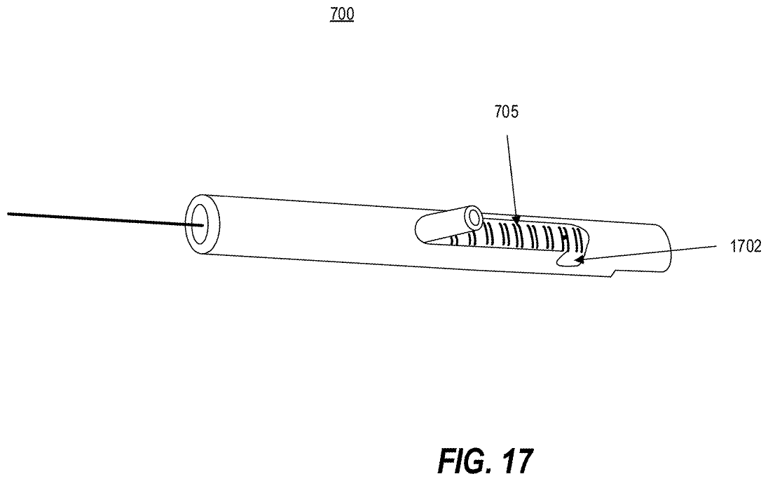

In some embodiments, the snare mechanism may be configured to enable moving the proximal ends of snare wire 108 and snare sleeve 107 together in a proximal and/or distal direction in order to move the distal end of snare 300 into and out of the second exit 110 of catheter 101 without changing the relative position of the distal end of snare sleeve 107 and snare tip 109. In other embodiments, the snare mechanism may be configured to allow moving the proximal end of snare wire 108 relative to the proximal end of snare sleeve 107 in order to move snare tip 109 relative to the distal end of snare sleeve 107, thereby exposing and/or covering snare tip 109 to enable grasping the distal tip 200 of guidewire 106. In some embodiments, as illustrated in FIG. 17, a snare mechanism, such as snare mechanism 700, may be spring-loaded such that a spring 705 can push snare sleeve 107 distally relative to snare tip 109, cause snare tip 109 to be covered, and lock onto the distal tip 200 of guidewire 106 once the distal tip 200 has been grasped. Additionally, or alternatively, the snare mechanism 700 may include a latch 1702 configured to latch snare sleeve 107 in its proximal position relative to snare tip 109 with snare tip 109 exposed and against the force of the spring 705. Latch 1702 may comprise a groove in the snare mechanism 700 and may be configured to receive and accommodate handle 706 of snare sleeve pusher 702 when snare sleeve pusher 702 is retracted. In some embodiments, the snare mechanism 700 may include a plurality of latches to lock snare tip 109 and/or snare sleeve 107 in particular locations relative to each other and/or relative to catheter 101. For example, the snare mechanism 700 may include one latch configured to accommodate handle 706 of snare sleeve pusher 702 and another latch configured to accommodate handle 707 of snare wire pusher 704 when snare sleeve pusher 702 and snare wire pusher 704 are retracted. In some embodiments, the snare mechanism 700 may be configured such that the snare mechanism 700 does not protrude from the handle of catheter 101 when maneuvering catheter 101. Accordingly, the snare mechanism 700 may not interfere with the manipulation of the handle of catheter 101.

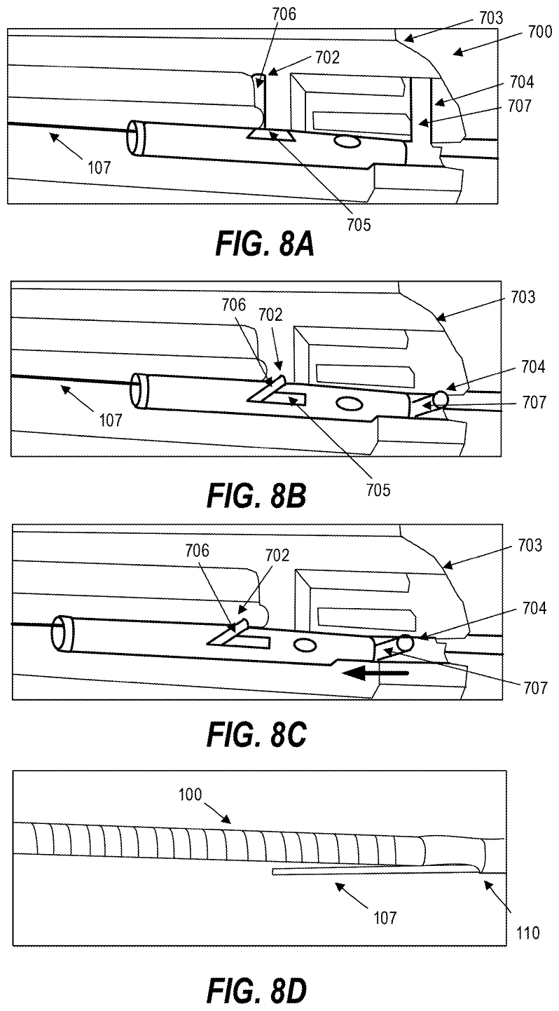

Referring now to FIGS. 8A-8D, the snare mechanism 700 may comprise a snare wire pusher 704 configured to slide in a proximal and/or distal direction within handle 703 of a catheter, such as catheter 101. The proximal end of a snare wire, such as snare wire 108 may be connected to snare wire pusher 704. The snare mechanism 700 may further comprise a snare sleeve pusher 702 located on snare wire pusher 704 and configured to slide in the proximal and/or distal direction relative to snare wire pusher 704. The proximal end of a snare sleeve, such as snare sleeve 107, may be connected to snare sleeve pusher 702. In some embodiments, snare sleeve pusher 702 may be coupled to a spring 705, and spring 705 may be configured to push snare sleeve pusher 702 in the distal direction relative to snare wire pusher 704. In some embodiments, snare wire pusher 704 may comprise a handle 707, which may be rotated between a storage position (as illustrated in FIG. 8A), in which handle 707 does not protrude from handle 703 of the catheter, and a manipulation position (as illustrated in FIG. 8B), in which handle 707 protrudes from handle 703. In yet another embodiment, snare sleeve pusher 702 may comprise a handle 706, which may be rotated when retracted in order to lock snare sleeve pusher 702 in its retracted position against the force of the spring. When handle 707 of snare wire pusher 704 is in its storage position (as illustrated in FIG. 8A), handle 706 of snare sleeve pusher 702 may also not protrude from handle 703 of the catheter.

FIGS. 8A-8D, 9A-9D, and 10A-10D illustrate an exemplary method of manipulating an exemplary snare mechanism 700, consistent with the embodiments of the present disclosure. By way of example, FIG. 8A illustrates snare mechanism 700 comprising a snare wire pusher 704 having a handle 707 and a snare sleeve pusher 702 having a handle 706 in handle 703 of catheter 101. The proximal end of a snare sleeve 107 may be connected to snare sleeve pusher 702. In some embodiments, snare sleeve pusher 702 may be coupled to a spring 705, and spring 705 may be configured to push snare sleeve pusher 702 in the distal direction relative to snare wire pusher 704. FIG. 8A illustrates snare mechanism 700 in a storage position, in which handle 707 of snare wire pusher 704 and handle 706 of snare sleeve pusher 702 do not protrude from handle 703 of catheter 101.

In some embodiments, snare mechanism 700 may be actuated from the storage position into a manipulation position. Referring now to FIG. 8B, snare mechanism 700 may be actuated into the manipulation position by rotating handle 707 of snare wire pusher 704 such that handle 704 is no longer in handle 703 of catheter 101, but rather protruding from handle 703 of catheter 101. After placing snare mechanism 700 into the manipulation position by rotating handle 707 of snare wire pusher 704, handle 707 of snare wire pusher 704 may be pushed distally (in the direction of the arrow in FIG. 8C). Upon pushing handle 707 of snare wire pusher 704 distally, a distal end of snare, such as snare sleeve 107 of snare 300, may protrude from a side lumen via the second exit 110 of catheter 101, as illustrated in FIG. 8D.

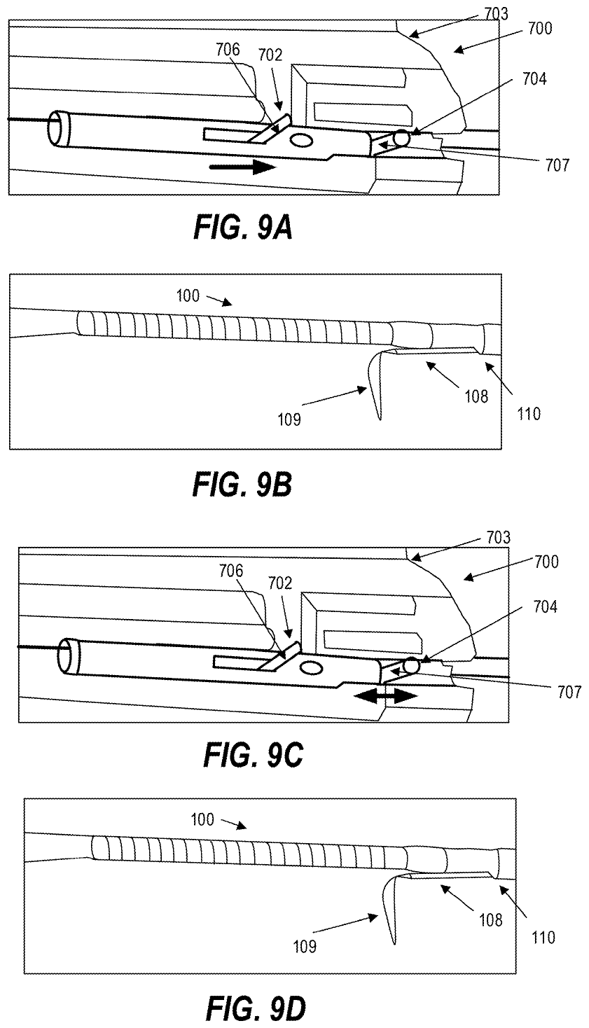

Referring now to FIGS. 9A-9D, in order to expose a snare tip 109 of snare 300, handle 706 of snare sleeve pusher 702 may be pulled proximally (in the direction of the arrow in FIG. 9A). As illustrated in FIG. 9B, pulling handle 706 proximally may expose snare tip 109 and snare wire 108 by retracting snare sleeve 107 and locking snare sleeve 107 in its retracted position. After exposing snare tip 109, handle 707 of snare wire pusher 704 may be controlled to adjust a position of snare tip 109 and facilitate passing guidewire 106 through snare tip 109. By way of example, as shown in FIG. 9C, handle 707 of snare wire pusher 704 may be moved distally and/or proximally in the direction of the arrow to reposition snare tip 109 in order to facilitate passing the distal tip 200 of guidewire 106 through snare tip 109. FIG. 9D illustrates snare tip 109 that is repositioned from FIG. 9B after moving handle 707 of snare wire pusher 704 distally.

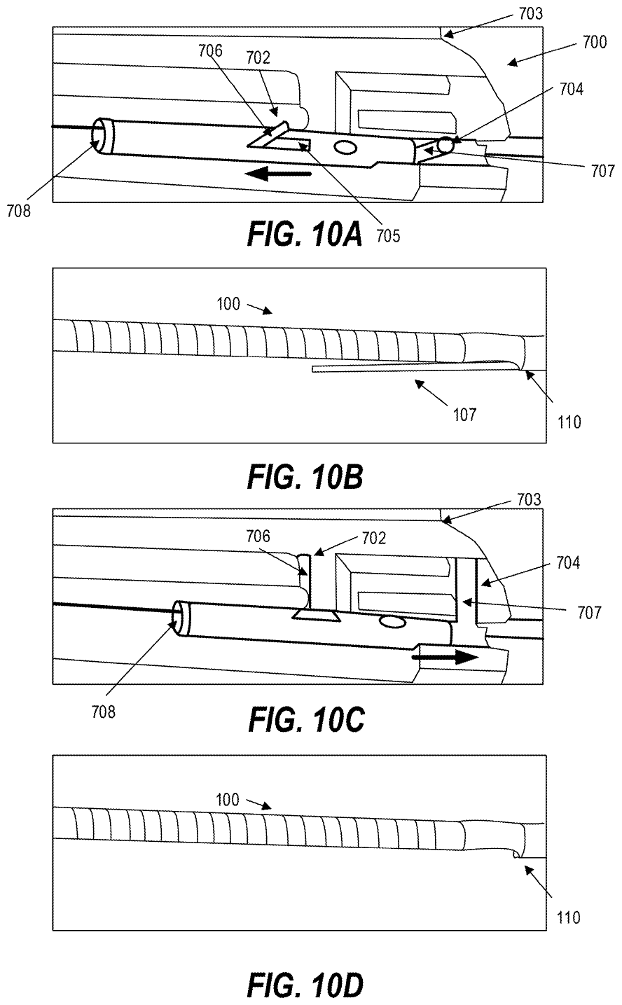

When the distal tip 200 of guidewire 106 is passing through snare tip 109, for example, handle 706 of snare sleeve pusher 702 may be released or moved in the direction of the arrow in FIG. 10A in order to allow spring 705 to push snare sleeve pusher 702 distally. As illustrated in FIG. 10B, when handle 706 of snare sleeve pusher 702 is released and spring 705 pushes snare sleeve pusher 702 distally, snare sleeve 107 may be configured to cover snare tip 109, thereby locking the distal tip 200 of guidewire 106 in snare tip 109. After covering snare tip 109 with snare sleeve 107, handle 707 of snare wire pusher 704 may be pulled proximally (in the direction of the arrow in FIG. 10C), thereby pulling snare sleeve 107 and distal tip 200 of guidewire 106 into the side lumen of catheter 101 via the second exit 110, as illustrated in FIG. 10D. Additionally, or alternatively, handle 707 of snare wire pusher 704 may be rotated back into the storage position as shown in FIG. 10C in order to lock snare 300 inside the side lumen of catheter 101.

In some embodiments of the present disclosure, a snare mechanism, such as snare mechanism 700, may be configured such that a snare wire and/or a snare sleeve can travel through one or more channels, such as channel 708 in FIGS. 10A and 10C, that provide support for the snare wire and/or the snare sleeve. Accordingly, the snare wire and/or the snare sleeve may not buckle when pushed distally. In some embodiments, the channels through which the snare wire and/or the snare sleeve travel may comprise an inner diameter that is smaller than about 10 mm in order to avoid the buckling of the snare wire or the snare sleeve. In some embodiments, for example, the channels may comprise an inner diameter that is smaller than about 6 mm. In some embodiments that channel may include an inner tube and an outer tube, and the outer tube may be configured to slide over the inner tube.

In other embodiments, a snare mechanism may comprise a snare sleeve pusher configured to slide in a proximal and/or distal direction within a handle of a catheter. In some embodiments, the proximal end of the snare sleeve may be connected to the snare sleeve pusher. Additionally, or alternatively, a snare wire pusher may be associated with the snare sleeve pusher and may be configured to slide in the proximal and/or distal direction relative to the snare sleeve pusher. In some embodiments, the proximal end of the snare wire may be connected to the snare wire pusher. The snare sleeve pusher and/or the snare wire pusher may comprise spring-loaded locking mechanisms that are configured to lock the snare sleeve pusher and/or the snare wire pusher in place and may be released, for example, by pressing a button. In some embodiments, the snare sleeve pusher may lock in place relative to the handle of the catheter, and the snare wire pusher may lock in place relative to the snare sleeve pusher. In other embodiments, when the snare sleeve pusher is repositioned, the snare wire pusher may move with the snare sleeve pusher.

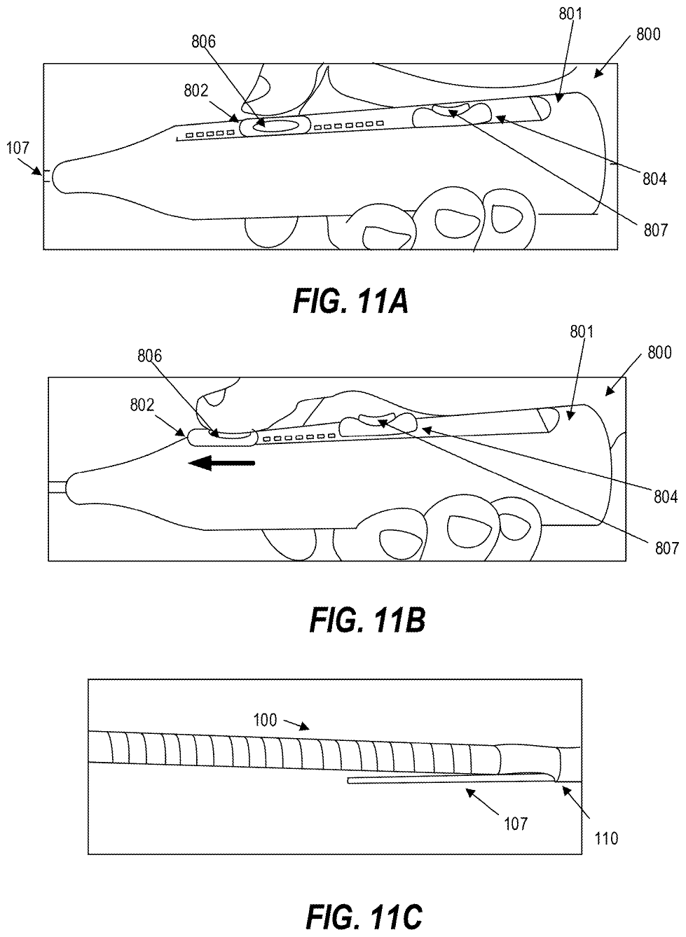

FIGS. 11A-11C, 12A-12B, 13A-13B, and 14A-14D illustrate an exemplary method of manipulating an exemplary snare mechanism 800, consistent with the embodiments of the present disclosure. By way of example, FIG. 11A illustrates snare mechanism 800 comprising a snare wire pusher 804 and a snare sleeve pusher 802 in handle 801 of a catheter, such as catheter 101. Snare sleeve pusher 802 may comprise a button 806 and snare wire pusher 804 may comprise a button 807. The proximal end of a snare sleeve, such as snare sleeve 107, may be connected to snare sleeve pusher 802. FIG. 11A illustrates snare mechanism 800 in a locked position. Accordingly, in the locked position, both snare wire pusher 804 and snare sleeve pusher 802 may be locked. In order to release snare sleeve pusher 802 from the locked position, button 806 of snare sleeve pusher 802 may be pushed, and snare sleeve pusher 802 may be moved distally in the direction of the arrow in FIG. 11B relative to the handle 801. Upon pushing button 806 to unlock snare sleeve pusher 802 and moving snare sleeve pusher 802 distally, as illustrated in FIG. 11C, a distal end of snare sleeve 107 (with snare tip 109 inside snare sleeve 107) may protrude from the side lumen of catheter 101 via the second exit 110.

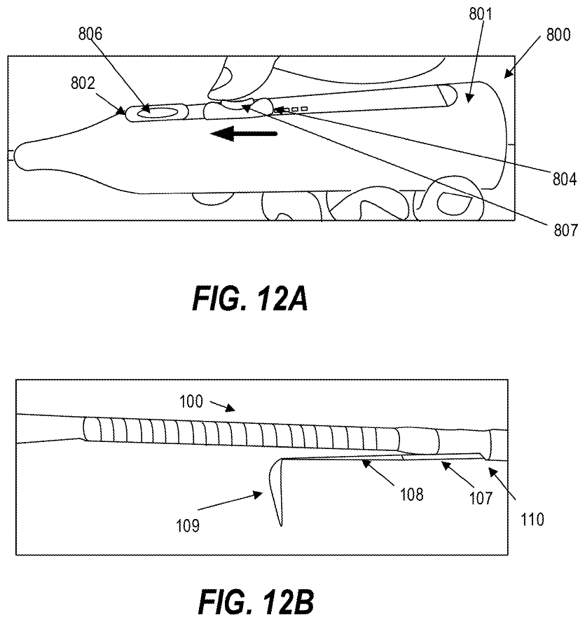

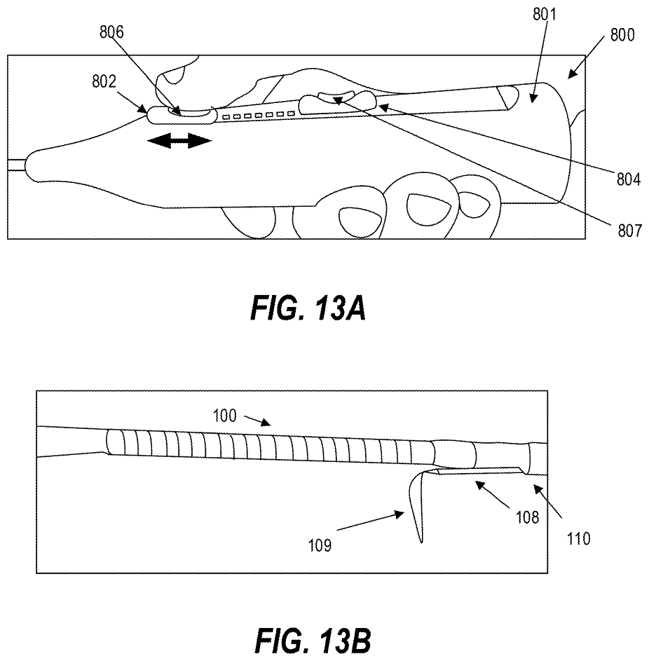

Referring now to FIGS. 12A and 12B, snare wire pusher 804 may also be released from its locked position. By way of example, in order to release snare wire pusher 804 from the locked position, button 807 of snare wire pusher 804 may be pushed, and snare wire pusher 804 may be moved distally in the direction of the arrow in FIG. 12A relative to snare sleeve pusher 802. Upon pushing button 807 to unlock snare wire pusher 804 and moving snare wire pusher 804 distally, snare tip 109 and snare wire 108 may be exposed from snare sleeve 107 as shown in FIG. 12B. Referring now to FIG. 13A, snare sleeve pusher 802 may be manipulated to position snare tip 109 to facilitate passing distal tip 200 of guidewire 106 through snare tip 109. By way of example, as illustrated in FIG. 13A, button 806 of snare sleeve pusher 802 may be pushed and snare sleeve pusher 802 may be moved distally and/or proximally in order to reposition snare tip 109 so as to facilitate passing distal tip 200 of guidewire 106 through snare tip 109. FIG. 13B, for example, illustrates snare tip 109 that is repositioned from FIG. 12B after moving snare sleeve pusher 802.

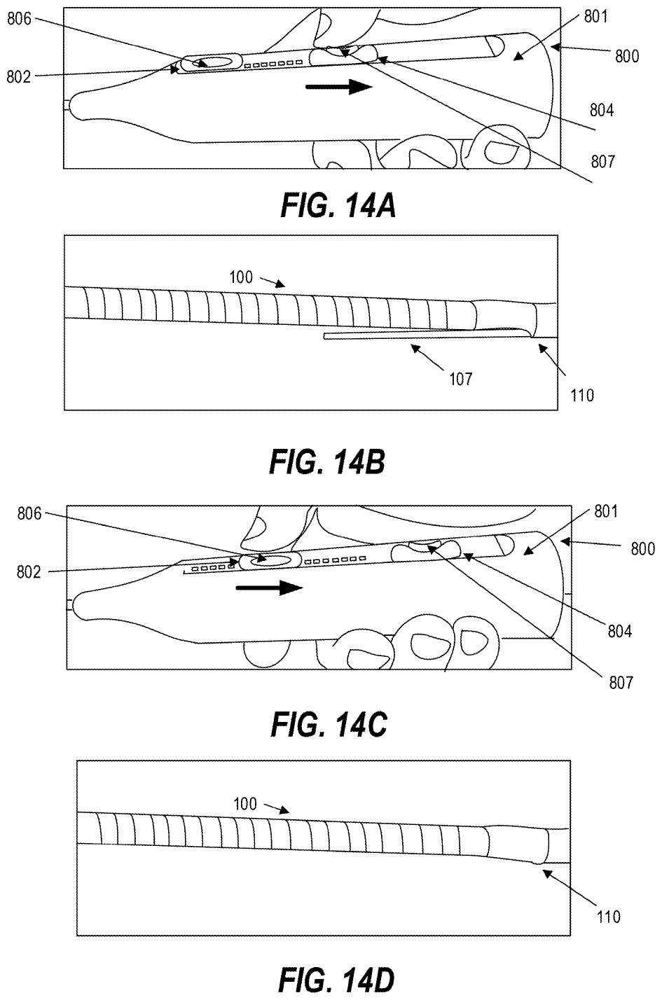

In some embodiments, when distal tip 200 of guidewire 106 is passing through snare tip 109, for example, button 807 of snare wire pusher 804 may be pushed in order to release its lock mechanism. Afterwards, as illustrated in FIG. 14A, snare wire pusher 804 may be moved proximally in the direction of the arrow in FIG. 14A relative to snare sleeve pusher 802 such that snare tip 109 can retract into snare sleeve 107, thereby locking distal tip 200 of guidewire 106 in snare tip 109. Accordingly, as shown in FIG. 14B, snare tip 109 may be retracted back into snare sleeve 107 after distal tip 200 of guidewire 106 passes through snare tip 109, thereby locking distal tip 200 of guidewire 106 in snare tip 109. In some embodiments, button 806 of snare sleeve pusher 802 may also be pushed to release its lock mechanism, and snare sleeve pusher 802 may be moved proximally in the direction of the arrow in FIG. 14C. Upon pushing snare sleeve pusher 802 proximally, snare sleeve 107 and the distal tip 200 of guidewire 106 within snare sleeve 107 may be pulled into the side lumen of catheter 101 via the second exit 110, as illustrated in FIG. 14D. In some embodiments, button 806 of snare sleeve pusher 802 may be released in order to lock snare tip 109 in the side lumen.

In some embodiments of the present disclosure, a distal portion of guidewire 106 that is proximal to and/or including distal tip 200 may be pre-shaped. By way of example, the distal portion of guidewire 106 may be pre-shaped into a curve such that when the distal portion exits the distal end of an inner catheter, such as inner catheter 105, the distal portion may curve towards the snare, such as snare 300, thereby facilitating snare 300 to grasp and capture distal tip 200 of guidewire 106. In some embodiments, the curved portion of guidewire 106 may have a predefined length such that a part of the curved portion remains inside inner catheter 105. Accordingly, the radius of curvature of the curved portion of guidewire 106 may be aligned with and/or correspond to the radius of curvature of the distal end of inner catheter 105. In some embodiments, the curved portion of guidewire 106 may be configured such that the entire length of the curved portion exits the inner catheter 105. Accordingly, guidewire 106 can be torqued so as to rotate the curved portion of guidewire 106 to an appropriate alignment and aid in capturing distal tip 200 of guidewire 106 with snare 300. Additionally, or alternatively, a distal portion of snare 300 and/or snare sleeve 107 may be pre-shaped. For example, a distal portion of snare 300 and/or snare sleeve 107 may be pre-shaped into a curve such that when the distal portion of snare 300 and/or snare sleeve 107 are exposed outside the second exit 110 of the side lumen, the distal portion of snare 300 and/or snare sleeve 107 may bend away from catheter 101, thereby facilitating snare 300 to grasp distal tip 200 of guidewire 106.

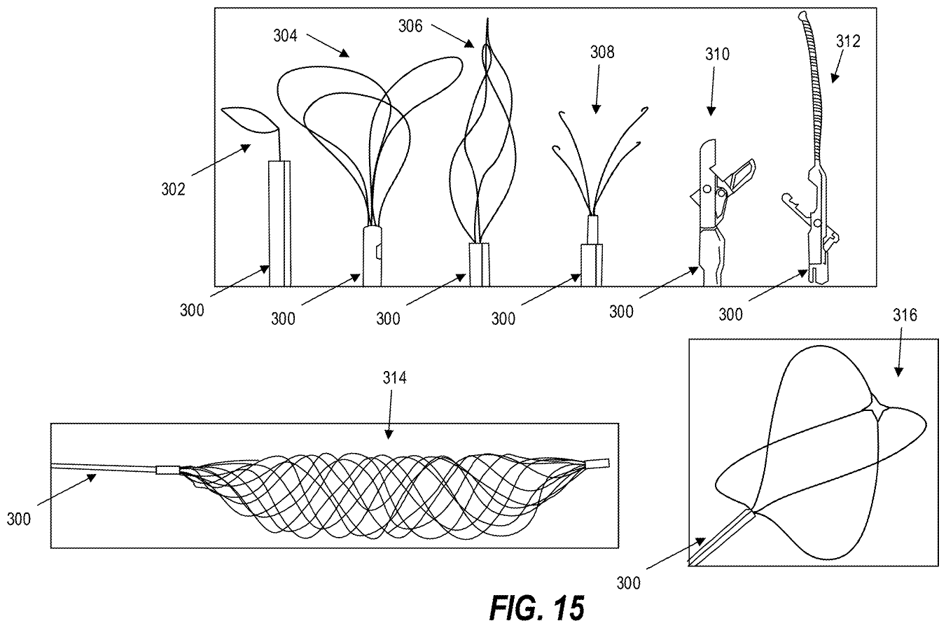

Referring now to FIG. 15, various embodiments of an exemplary snare, such as snare 300, are provided. As discussed above, the term “snare” may generally refer to any mechanism that is capable of being mounted on a wire or a thin tube and that can be used to capture and grasp an end of a guidewire. As illustrated in FIG. 15, in some embodiments, a snare, such as snare 300, may include at least one of one or more wires 308, one or more coil loops 302, 304, 306, 316, a braid 314, or a grasper 310, 312. Additionally, or alternatively, a snare, such as snare 300, may include a basket, a funnel, a magnet, or any other mechanism configured to capture an end of a guidewire.

While the present disclosure is described herein with reference to illustrative embodiments of catheters used for particular applications, such as for navigation through a ventricle of a heart for cardiac repair, it should be understood that the embodiments described herein are not limited thereto. Those having ordinary skill in the art and access to the teachings provided herein will recognize additional modifications, applications, embodiments, and substitution of equivalents that all fall within the scope of the disclosed embodiments. Accordingly, the disclosed embodiments are not to be considered as limited by the foregoing or following descriptions.

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described and accordingly, all suitable modifications and equivalents may be resorted to, falling within the scope of the present disclosure.

Moreover, those skilled in the art will appreciate that the conception upon which this disclosure is based may readily be used as a basis for designing other structures, methods, and systems for carrying out the several purposes of the present disclosure. Accordingly, the claims are not to be considered as limited by the foregoing description.

1. A device for passing a guidewire around an anatomical structure of interest within a body, the device comprising:
a catheter including an elongated tube having at least a first lumen and a second lumen;
a guidewire configured to pass through the first lumen of the catheter; and
a snare configured to pass through the second lumen of the catheter;
wherein a first exit of one of the first lumen and the second lumen is positioned at or near a distal tip of the catheter,
wherein a second exit of another of the first lumen and the second lumen is positioned on a side of the catheter at a distance proximal to a distal tip of the catheter, and
wherein a portion of the catheter distal to the second exit is configured to loop around at least a portion of the anatomical structure of interest within the body and towards the second exit.

2. The device of claim 1, wherein the first lumen is a central lumen of the catheter with the first exit positioned at or near the distal tip of the catheter, and wherein the second lumen is a side lumen of the catheter with the second exit positioned on the side of the catheter at the distance proximal to the distal tip of the catheter.

3. The device of claim 1, wherein the second lumen is a central lumen of the catheter with the first exit positioned at or near the distal tip of the catheter, and wherein the first lumen is a side lumen of the catheter with the second exit positioned on the side of the catheter at the distance proximal to the distal tip of the catheter.

4. The device of claim 1, wherein the second exit is positioned between about 2 cm and about 20 cm proximal to the distal tip of the catheter.

5. The device of claim 1, wherein the second exit is positioned between about 5 cm and about 15 cm proximal to the distal tip of the catheter.

6. The device of claim 1, wherein the portion of the catheter distal to the second exit includes at least one of a pre-shaped portion, a flexible portion, a steerable portion, or a portion with bending properties.

7. The device of claim 1, wherein the portion of the catheter distal to the second exit includes at least one of a metal wire braid, a metal wire coil, a cut hypotube, a non-metal braid, a pullwire, a pre-shaped polymer, or a pre-shaped shape-memory component.

8. The device of claim 1, wherein a portion of the catheter between the first exit and the second exit is configured to position the first exit and the second exit such that the guidewire and the snare exiting the first and second lumens engage each other.

9. The device of claim 1, wherein the snare is pre-loaded within the second lumen of the catheter, and wherein a handle of the catheter includes a mechanism for exposing, positioning, and retracting the snare.

10. The device of claim 9, wherein the snare includes a snare sleeve and a snare wire within the snare sleeve, and wherein the mechanism for exposing, positioning, and retracting the snare includes a first slider configured to control a movement of the snare sleeve and a second slider configured to control a movement of the snare wire.

11. The device of claim 9, wherein the mechanism for exposing, positioning, and retracting the snare includes a sliding U-shaped tube.

12. The device of claim 1, wherein the guidewire is pre-loaded within the first lumen of the catheter, and wherein a handle of the catheter includes a mechanism for storing a length of the guidewire within the handle.

13. The device of claim 1, wherein a distal portion of the guidewire is pre-shaped to facilitate engaging a distal end of the guidewire with the snare.

14. The device of claim 1, wherein the snare includes at least one of a snare sleeve or a snare wire, and wherein a distal portion of at least one of the snare wire or the snare sleeve is pre-shaped to facilitate engaging a distal end of the guidewire with the snare.

15. The device of claim 1, further comprising an inner catheter configured to pass through a central lumen of the catheter, wherein:
the guidewire or the snare is configured to pass through a lumen of the inner catheter,
a distal end of the inner catheter extends past the distal tip of the catheter, and
the distal end of the inner catheter is configured to aid in positioning a distal end of the guidewire or the snare.

16. The device of claim 15, wherein the distal end of the inner catheter is pre-shaped.

17. The device of claim 15, wherein the distal end of the inner catheter is steerable.

18. The device of claim 9, wherein the mechanism for exposing, positioning, and retracting the snare is configured such that the snare wire travels through a channel with an inner diameter of less than 10 mm.

19. The device of claim 9, wherein the mechanism for exposing, positioning, and retracting the snare is configured such that the snare wire travels through a channel with an inner diameter of less than 6 mm.

20. The device of claim 9, wherein the mechanism for exposing, positioning, and retracting the snare is configured such that the snare wire travels through a channel comprising an outer tube and an inner tube, and wherein the outer tube is configured to slide over the inner tube.
